(19) United States
(12) Patent Application Publication
(10) Pub. No.: US 2024/0125707 A1
(43) Pub. Date: Apr. 18, 2024

(54) DIFFRACTOMETRIC SENSOR FOR THE DETECTION OF BINDING AFFINITIES

(71) Applicants: HOFFMANN-LA ROCHE INC., Little Falls, NJ (US); EIDGENOESSISCHE TECHNISCHE HOCHSCHULE ZUERICH, Zuerich (CH)

(72) Inventors: Christof FATTINGER, Zuerich (CH); Andreas FRUTIGER, Zuerich (CH); Andreas Michael REICHMUTH, Zuerich (CH); Yves Lukas BLICKENSTORFER, Zuerich (CH); Janos VOEROES, Zuerich (CH); Roman POPOV, Zuerich (CH)

(21) Appl. No.: 18/254,368

(22) PCT Filed: Nov. 26, 2021
(86) PCT No.: PCT/EP2021/083109

§ 371 Date: May 24, 2023

(30) Foreign Application Priority Data
Nov. 27, 2020 (EP) 20210192.9

(51) Int. Cl. G01N 21/77; G01N 21/47; G01N 33/543

(52) U.S. Cl. CPC G01N 21/7743; G01N 21/4788; G01N 33/54373; G01N 2021/7779

(57) ABSTRACT

A diffractometric sensor (1), comprises:—a substrate (3);—two interdigitated affinity gratings (2), a first affinity grating (20) comprising first unit cells (200) with affinity elements (201) and a second affinity grating (21) comprising second unit cells (210) with affinity elements (211), wherein the first unit cells (200) and second unit cells (210) are configured and arranged such that coherent light of a predetermined wavelength generated at a predetermined beam generation location (40) and diffracted by target molecules (204, 214)) bound to the affinity elements (201, 211) constructively interferes at a predetermined detection location (50) with an inverse phase, and wherein the first and second affinity gratings (20, 21) are balanced to generate a bias signal at the predetermined detection location (50) that corresponds to a difference (Am) in the scattering mass of the first and second affinity gratings (20, 21) which is in the range of 0.001 pg/mm² to 30000 pg/mm².

### FIELD

The present invention relates to a diffractometric sensor for the detection of binding affinities.

### BACKGROUND

In the detection or monitoring of binding affinities (for example, when the presence of a target molecule contained in a sample is to be detected) interaction sensors are oftentimes used in a large variety of types and applications. By way of example, for detecting the affinity of target molecules or a group of target molecules that share a common property or binding site to bind to affinity elements, a large number of affinity elements of a specific kind are immobilised on the outer surface of the interaction sensor. An affinity element denotes any kind of element or moiety of element that leads to either an attachment or to a predetermined minimum residence time of a particular target molecule or group of target molecules on the interaction sensor (the minimum residence time must at least be sufficiently long to allow for reliable detection of the target molecules or group of target molecules). The binding (or non-binding) of the target molecules to this specific kind of affinity elements is detected and is used to provide information on the binding affinity of the target molecules or group of target molecules that share a common property or binding site with respect to this specific kind of affinity elements.

Important applications of interaction sensors involve the detection or monitoring of the binding affinities of target molecules in samples that contain a large amount and a vast diversity of background molecules that might interfere with the binding affinities of the target molecules. In state of the art interaction sensors, washing of the sensor surface is performed to minimize interferences of background molecules in the detection or monitoring of binding affinities of the target molecules.

One type of interaction sensor for detecting binding affinities utilizes fluorescent labels attached to the target molecules.

The fluorescent labels are capable of emitting fluorescent light upon excitation. The emitted fluorescent light has a characteristic emission spectrum which is indicative of the presence of the fluorescent label on the sensor. The presence of the fluorescent label in turn indicates that the labelled target molecules have bound to the affinity elements on the sensor.

A sensor for detecting labelled target molecules is described in the article “Zeptosens' protein microarrays: A novel high performance microarray platform for low abundance protein analysis”, Proteomics 2002, 2, S. 383-393, Wiley-VCH Verlag GmbH, 69451 Weinheim, Germany. The sensor described there comprises a planar waveguide arranged on a substrate. The planar waveguide has an outer surface having a plurality of binding sites (affinity elements) thereon. Moreover, the planar waveguide has a plurality of grating lines for coupling a beam of coherent light into the planar waveguide in a manner such that the coherent light coupled into the planar waveguide propagates along the waveguide. The coherent light propagates through the planar waveguide under total reflection, with an evanescent field of the coherent light propagating along the outer surface of the planar waveguide. The depth of penetration of the evanescent field into the medium of lower refractive index at the outer surface of the planar waveguide is in the order of magnitude of a fraction of the wavelength of the coherent light propagating through the planar waveguide. The evanescent field excites the fluorescent labels of the labelled target molecules bound to the binding sites (affinity elements) arranged on the surface of the planar waveguide. Due to the very small depth of penetration of the evanescent field into the optically thinner medium at the outer surface of the planar waveguide, only the labelled target molecules bound to the binding sites (affinity elements) on the outer surface of the planar waveguide are excited. The fluorescent light emitted by the labels is then detected with the aid of a CCD camera.

While it is principally possible to detect binding affinities using fluorescent labels, this technique is disadvantageous in that the detected signal is produced by fluorescent labels rather than by the binding partners themselves. In addition, labelling the target molecules requires additional preparation steps.

Moreover, labelled target molecules are comparatively expensive.

Another disadvantage is the falsification of the results caused by steric hindrance of the fluorescent labels at the target molecules which might interfere with the binding of the target molecules to the binding sites (affinity elements). Further disadvantages are the falsification of the results due to photobleaching of the labels or quenching effects.

Another type of interaction sensors are refractometric sensors, i.e. sensors that generate a signal based on a change in refractive index (RI) caused by target molecules bound to affinity elements on the interaction sensor. The Figure Of Merit (FOM_{RI}) of refractometric sensors is in the order of 100-1000 per Refractive Index Unit (RIU), i.e. 100-1000/RIU. The Figure of Merit (FOM_{RI}) connects the measurement accuracy (represented by the standard deviation σᵣₑₗ of the normalized sensor output signal, for example of an intensity measurement) to the refractive index resolution (represented by the standard deviation σ_{RI} of the refractive index resolution) of the sensor according to the equation:

σ_{RI}=σᵣₑₗ/FOM_{RI}

In other words, the lower the Figure Of Merit (FOM_{RI}) of the refractometric sensor, the higher the required measurement accuracy σᵣₑₗ of the normalized sensor output signal to achieve the same refractive index resolution σ_{RI}. With state of the art refractometric interaction sensors refractive index resolutions σ_{RI} in the order of 10⁻⁷ RIU can be achieved.

Consequently, with FOM_{RI} in the range of 100-1000/RIU the required measurement accuracy σᵣₑₗ is in the order of 10⁻⁵. Such measurement accuracy can only be achieved with very expensive scientific measurement equipment, if at all.

Also, after the target molecules have been applied to the surface of the sensor to bind to the affinity elements and prior to the detection, an extensive washing of the surface of the sensor must be performed in order to wash off background molecules from the surface of the sensor that have not bound to the affinity elements of the sensor. One consequence of the extensive washing process is that target molecules that only weakly bind to the affinity elements of the sensor cannot be detected at all, since even in case they weakly bind to the affinity elements they are washed off during the washing process.

### SUMMARY OF INVENTION

It is an object of the invention to suggest a sensor that does not exhibit the above-mentioned disadvantages associated with refractometric interaction sensors, and in particular it is an object to suggest an interaction sensor having a significantly higher Figure of Merit (FOM) so that a reliable detection of extremely small quantities of a target molecule is possible using simple and inexpensive measurement equipment. Also the sensor should be capable of detecting target molecules in samples that contain a large amount and a vast diversity of background molecules. Also, the sensor should be capable of detecting target molecules that bind only weakly to the affinity elements of the sensor.

In accordance with the present invention, this object is achieved with a completely new type of sensor, in the following called a ‘diffractometric sensor’. A diffractometric sensor is specified by the features of the independent claim. Advantageous aspects of the diffractometric sensor according to the invention are the subject of the dependent claims.

The diffractometric sensor according to the invention comprises:
a substrate;
two interdigitated affinity gratings arranged on the substrate, a first affinity grating and a second affinity grating.

The first affinity grating comprises first unit cells and the second affinity grating comprising second unit cells.

The first unit cells of the first affinity grating comprises affinity elements of a first type capable of binding with target molecules of a first type, and the second unit cells of the second affinity grating comprising affinity elements of a second type capable of binding with target molecules of a second type.

The first unit cells of the first affinity grating are configured and arranged such that coherent light of a predetermined wavelength generated at a predetermined beam generation location and diffracted by target molecules of the first type bound to the affinity elements of the first type constructively interferes at a predetermined detection location with a first phase.

The second unit cells of the second affinity grating are configured and arranged such that the coherent light of the predetermined wavelength generated at the predetermined beam generation location and diffracted by target molecules of the second type bound to the affinity elements of the second type constructively interferes at the predetermined detection location with a second phase inverse to the first phase.

The first and second affinity gratings are balanced with respect to a scattering mass of the first and second affinity gratings to generate a bias signal at the predetermined detection location that corresponds to a difference in the scattering mass of the first and second affinity gratings which is in the range of 0.001 pg/mm² to 30000 pg/mm².

An affinity grating is a grating that comprises affinity elements, i.e. elements which are capable of binding a specific target molecule or a specific type of target molecules. ‘Type’ of target molecules means that all target molecules of the same ‘type’ have a common property or moiety (e.g. a binding site) that allows these target molecules to bind to the same affinity element.

The affinity elements are arranged in unit cells of the respective affinity grating, i.e. the first unit cells comprise affinity elements of a first type capable of binding with a first type of target molecules, and the second unit cells comprise affinity elements of a second type capable of binding with a second type of target molecules. The first type of affinity elements and the second type of affinity elements may be identical or may be different. In many applications, the first type of affinity elements and the second type of affinity elements are different and, accordingly, the first type of target molecules that may bind to the first type of affinity elements and the second type of target molecules that may bind to the second type of affinity elements are different, too.

The first unit cells of the first affinity grating are configured and arranged such that coherent light of a predetermined wavelength generated at a predetermined beam generation location and diffracted by target molecules of the first type bound to the affinity elements of the first type constructively interferes at a predetermined detection location with a first phase. The term ‘predetermined wavelength’ denotes the wavelength of the coherent light which must be known in advance and which is typically a single wavelength (meaning that the coherent light is monochromatic). The term ‘predetermined beam generation location’ denotes the location where the beam of coherent light is generated, and must also be known in advance.

Of course, in case the sensor is tunable (for example within very small ranges as regards the exact location of the light source, as regards the direction of impingement of the beam of coherent light, as regards the predetermined wavelength of the coherent light, or as regards the exact location of the detector), the predetermined beam generation location is allowed to vary to an extent such that it is within the tuning range of the sensor. Similarly, the term ‘predetermined detection location’ denotes the location where the coherent light diffracted by the first type of target molecules bound to the affinity elements of the first type is detected, and must also be known in advance. Again, in case the sensor is tunable, the predetermined detection location may vary to an extent such that it is within the tuning range of the sensor. Only in case the coherent light has the predetermined wavelength, the beam of coherent light of this predetermined wavelength is generated at the predetermined beam generation location, and the coherent light diffracted by the first type of target molecules bound to the affinity elements of the first type is detected at the predetermined detection location, the diffracted light constructively interferes at the detection location with a first phase.

The second unit cells of the second affinity grating are configured and arranged such that the coherent light of the predetermined wavelength generated at the predetermined beam generation location and diffracted by target molecules of the second type bound to the affinity elements of the second type constructively interferes at the predetermined detection location with a second phase. However, the second affinity grating is configured such that this second phase is inverse to the first phase.

Due to these inverse phases, the two interdigitated affinity gratings represent an optical comparator that measures the difference in diffraction efficiency of the two interdigitated gratings (i.e. the first affinity grating and the second affinity grating). The difference in diffraction efficiency is proportional to the square of the difference in scattering mass per unit area Γ_{b} (e.g. pg/mm², picograms per square millimeter).

The scattering mass is the mass density of the spatial Fourier component of the mass density distribution that fulfils the diffraction condition of the grating. All Fourier components of the mass density that do not fulfil the diffraction condition are not detectable by the sensor. This allows for the detection or monitoring of binding affinities of target molecules in samples that contain a large amount and a vast diversity of background molecules that might interfere with the binding affinities of the target molecules in a wash-free and real-time format, i.e. it allows for wash-free and real-time immunoassays. This is because the non-specific binding of background molecules is diluted over a large spectrum in Fourier space and is thus not detectable by the sensor.

The Figure of Merit (FOM_{diff}) of such diffractometric sensor is inversely proportional to this difference in scattering mass per unit area Γ_{b} according to the equation:

FOM_{diff}=2/Γ_{b}

From this equation it follows that the smaller the difference in scattering mass per unit area Γ_{b} (and hence also the diffraction efficiency) is (at the time of performing the measurement), the higher is the Figure of Merit (FOM_{diff}) of the diffractometric sensor, and the lower is the required accuracy of the measurement equipment.

Generally, this suggests that the difference in diffraction efficiency (i.e. the difference in scattering mass per unit area) of the first affinity grating and the second affinity grating be selected as small as possible at the time of measurement, ideally zero, as this is suggested in US 2015/0276612. On the other hand, even an optimally balanced diffractometric sensor (i.e. a diffractometric sensor which is perfectly balanced with respect to the scattering mass of the two interdigitated affinity gratings) arranged at the predetermined detection location generates a small background signal (speckles) that corresponds to a certain difference in scattering mass (diffraction efficiency) between the first and second affinity gratings.

Accordingly, the problem here is that the minimum mass of target molecules that can be reliably detected by the diffractometric sensor should be as small as possible, while at the same time it should be avoided that the signal generated by such minimum detectable mass of target molecules be interpreted as a speckle (avoidance of false negatives). On the other hand, it must be avoided that a speckle be inadvertently interpreted as a signal caused by the minimum detectable mass of target molecules (avoidance of false positives).

As already explained, the Figure of Merit (FOM_{diff}) of the diffractometric sensor is inversely proportional to the difference in scattering mass per unit area (whereas the Figure of Merit (FOM_{RI}) of a refractometric sensor is independent thereof). Or more frankly speaking, the Figure of Merit (FOM_{diff}) of the diffractometric sensor is inversely proportional to the bias signal generated by the detector arranged at the detection location so that selecting too high a bias signal would lead to a Figure of Merit (FOM_{diff}) of the diffractometric sensor requiring a measurement accuracy higher than the measurement accuracy required for a refractometric sensor.

Therefore, the interdigitated first and second affinity gratings of the diffractometric sensor according to the invention are balanced with respect to a difference in scattering mass of the first and second affinity gratings such that they generate a bias signal at the predetermined detection location that corresponds to a difference in the scattering mass of the first and second affinity gratings which is in the range of 0.001 pg/mm² to 30000 pg/mm², depending on the particular embodiment and application of the diffractometric sensor (as will be explained in more detail below).

Just to give a quick example illustrating the advantages of a diffractometric sensor according to the invention vis-a-vis a prior art refractometric sensor:
The Figure of Merit FOM_{RI} of known refractometric sensors may be about FOM_{RI}=100/RIU (RIU=Refractive Index Unit).
The Figure of Merit FOM_{diff} of a diffractometric sensor—after conversion to inverse RIUs using the De Feijter formula—may be about FOM_{diff}=5·10⁵/RIU.

As the measurement accuracy σᵣₑₗ and the Figure of Merit (FOM) are linked according to σ_{RI}=σᵣₑₗ/FOM (see further above), assuming that a refractive index resolution of σ_{RI}=10⁻⁷ is to be achieved, the required measurement accuracy of a refractometric sensor must be σᵣₑₗ=σ_{RI}·FOM_{RI}=0.00001, i.e. 10⁻⁵ (or 0.001%). Such measurement accuracy can be achieved, if at all, using extremely expensive scientific measurement equipment. With a diffractometric sensor according to the invention, the required measurement accuracy is σᵣₑₗ=σ_{RI}·FOM_{diff}=0.05, i.e. 5·10⁻² (or 5%), which can be easily achieved using simple and inexpensive measurement equipment. This renders the diffractometric sensor extremely advantageous.

In some embodiments of the diffractometric sensor according to the invention, the bias signal corresponding to the difference in the scattering mass of the first and second affinity gratings is in the range of 0.1 pg/mm² to 1000 pg/mm², more particularly in the range of 0.1 pg/mm² to 100 pg/mm², and even more particularly in the range of 1 pg/mm² to 10 pg/mm². These ranges represent advantageous sub-ranges for practical embodiments of the diffractometric sensor according to the invention.

In some embodiments of the diffractometric sensor according to the invention, the concentration or the spatial arrangement of the affinity elements of the first type in the first unit cells and the concentration or the spatial arrangement of the affinity elements of the second type in the second unit cells (of the interdigitated affinity gratings) are different. A ‘different concentration’ denotes a different number of affinity elements in the first and second unit cells of same volume, whereas a ‘different spatial arrangement’ is intended to cover cases in which the number of affinity elements in the first and second unit cells may be the same, but the spatial distribution of the affinity elements in the first and second unit cells is significantly different.

This difference in the concentration or spatial arrangement of the affinity elements is generally independent of whether the affinity elements of the first type in the first unit cells and the affinity elements of the second type in the second unit cells are identical or different. However, in one embodiment of the diffractometric sensor the affinity elements of the first type in the first unit cells and the affinity elements of the second type in the second unit cells are identical. In case target molecules bind to this (identical) type of affinity elements, due to the different concentration or due to the different spatial arrangement of the affinity elements in the first and second unit cells, different amounts (mass) of target molecules are bound to the first unit cells and to the second unit cells.

For example, the spatial distribution of the affinity elements in the first and second unit cells may be such that two or more affinity elements in the first unit cells bind to the same target molecule, whereas in the second unit cells each affinity element binds to one target molecule. As a consequence thereof, the total mass of target molecules bound to the affinity elements of the first unit cells is smaller than the total mass of target molecules bound to the affinity elements of the second unit cells. In addition, when two affinity elements bind to a target molecule the affinity is higher than the added affinities of two individual affinity elements, i.e. there are synergistic binding or avidity effects. Since the target molecules bound to the affinity elements of both the first unit cells and the second unit cells diffract the coherent light to the detection location, however, with inverse phases, a differential signal is generated at the detection location which may be detected and used to determine the presence of a target molecule.

In some embodiments of the diffractometric sensor according to the invention, the affinity elements of the first type are non-binding (inert) for the target molecules of the second type or the affinity elements of the second type are non-binding (inert) for the target molecules of the first type, or both (i.e. the affinity elements of the first type are non-binding for the target molecules of the second type and the affinity elements of the second type are non-binding for the target molecules of the first type). In addition, the first type of affinity elements and the second type of affinity elements preferably allow for a similar, preferably minimal, capability of binding to background molecules (i.e. molecules other than target molecules).

In practice, target molecules—while preferentially binding to one type of affinity element—often also bind to other types of affinity elements (but with considerably smaller affinity). Such scenario is included when stating that an affinity element of a first type is non-binding for target molecules of a second type, and vice versa (since a perfect affinity element does not exist in this regard).

In some embodiments of the diffractometric sensor according to the invention, at least one of the two interdigitated affinity gratings (that is to say only one of the two interdigitated gratings or both of the two interdigitated gratings) further comprises binding sites capable of binding scattering elements.

Such binding sites capable of binding scattering elements may be arranged in or on the substrate, but in particular they may be arranged in the unit cells. Such binding sites capable of binding scattering elements essentially do not increase the bias signal, however, they allow scattering elements to be added which may bind to these binding sites capable of binding the scattering elements. Adding the scattering elements may be performed before or during an assay.

In some further embodiments of the diffractometric sensor according to the invention, at least one of the two interdigitated affinity gratings (that is to say only one of the two interdigitated gratings or both of the two interdigitated gratings) further comprises scattering elements. Like the binding sites capable of binding the scattering elements, the scattering elements may generally be arranged in or on the substrate, but in particular they may be arranged in the unit cells. In some embodiments (those embodiments comprising binding sites capable of binding the scattering elements), the scattering elements are bound to the binding sites capable of binding the scattering elements. In other embodiments (not comprising binding sites capable of binding the scattering element) the scattering elements may be arranged in or on the substrate or in the unit cells. The term ‘scattering elements’ is to be understood to denote elements (other than target molecules) changing the scattering power or scattering strength of the unit cells of the affinity gratings.

In some embodiments of the diffractometric sensor according to the invention, the scattering elements are tunable or cleavable to allow for adjustment of the scattering power/strength or removal of the scattering elements. ‘Tunable’ means that the scattering power/strength of the scattering elements can be changed. As an example, the scattering power/strength of the scattering elements may be changed through the application of an external electric field in case the scattering element is made from an electrooptic material. ‘Cleavable’ means that the scattering elements may be removable. Such change of the scattering power/strength (or even cleavage/removal of scattering elements) may be helpful in adjusting the bias signal.

In some embodiments of the diffractometric sensor according to the invention the two interdigitated affinity gratings are arranged on a surface of the substrate. Generally, in such embodiments the predetermined beam generation location and the predetermined detection location may be arranged on the same side of the substrate (e.g. the predetermined beam generation and the predetermined detection location both are arranged above or below the substrate) or on different sides of the substrate (e.g. one is located above the substrate while the other one is located below the substrate). According to a further aspect, such diffractometric sensor may further comprise an optical coupler configured and arranged to direct the coherent light coming from the predetermined beam generation location to the two interdigitated affinity gratings arranged on the surface of the substrate. And according to yet a further aspect, such diffractometric sensor may further comprise an optical decoupler configured and arranged to direct the coherent light diffracted by the two interdigitated affinity gratings to the predetermined detection location.

In other embodiments, the diffractometric sensor according to the invention may further comprise a resonant waveguiding structure arranged on the surface of the substrate. The resonant structure is configured to allow for coupling of the coherent light of the predetermined wavelength generated at the predetermined beam generation location into the resonant waveguiding structure to generate an evanescent field propagating along an outermost surface of the resonant waveguiding structure opposite to a surface of the resonant waveguiding structure facing the substrate. The two interdigitated affinity gratings are arranged on the outermost surface of the resonant waveguiding structure.

Such resonant waveguiding structures include, in one embodiment, multi-layer resonant structures comprising a stack of dielectric layers of materials having a different refractive index, alternatingly arranged one above the other (i.e. layer of higher refractive index, layer of lower refractive index, layer of higher refractive index, layer of lower refractive index, etc.) which allow for the generation of Bloch surface waves at the outermost surface opposite to the surface of facing the substrate. For example, in a vertically stacked arrangement of such dielectric layers, the coherent light of the predetermined wavelength may be coupled into the lowermost layer, which will then be coupled into the adjacent layer arranged immediately above the lowermost layer, and so on, until reaching the uppermost layer. The coherent light coupled into the said uppermost layer propagates within this uppermost layer, with an evanescent field thereof propagating along the uppermost surface of this uppermost layer. The two interdigitated affinity gratings are then arranged on this uppermost surface.

In other embodiments of the diffractometric sensor according to the invention, the resonant waveguiding structure arranged on the surface of the substrate may be a (single) planar waveguide, and the two interdigitated affinity gratings are arranged on a surface of the planar waveguide opposite to a surface of the planar waveguide facing the substrate. Also here, the coherent light coupled into the planar waveguide propagates along the planar waveguide, with an evanescent field thereof propagating along the surface of the planar waveguide opposite to the surface of the planar waveguide facing the substrate. On this surface of the planar waveguide opposite to the surface facing the substrate, the two interdigitated affinity gratings are arranged.

In some further embodiments of the diffractometric sensor according to the invention, the planar waveguide is structured so as to guide the coherent light of the predetermined wavelength generated at the beam generation location and coupled into the planar waveguide in one or more predetermined directions along the surface of the planar waveguide opposite to the surface facing the substrate. Such structured waveguides allow for guiding the coherent light in practically any desired direction so that this configuration may be advantageous in forming so-called photonic integrated circuits (PICs).

In yet further embodiments of the diffractometric sensor according to the invention, the sensor may further comprise an optical coupler (e.g. a coupling grating) arranged on the planar waveguide and configured to couple the beam of coherent light generated at the beam generation location into the planar waveguide to impinge on the two interdigitated affinity gratings. And in still further embodiments of the diffractometric sensor according to the invention, the sensor may further comprise an optical decoupler (e.g. a decoupling grating) arranged on the planar waveguide and configured to decouple the coherent light diffracted by the two interdigitated affinity gratings from the planar waveguide and direct it to the predetermined detection location.

In some further embodiments of the diffractometric sensor according to the invention, the sensor may further comprise a detector for detecting the coherent light diffracted by the two interdigitated affinity gratings, with the detector being integrated in the planar waveguide or in the substrate. In such embodiment the detector is already arranged at the predetermined detection location, so that the sensor must only be correctly positioned relative to the beam generation location, any positioning of the detector relative to the two interdigitated affinity gratings is no longer needed. And in yet some further embodiments of the diffractometric sensor according to the invention, the sensor may further comprise a light source for generating the beam of coherent light of the predetermined wavelength, with the light source being integrated in the planar waveguide or in the substrate. In such embodiment the light source is already arranged at the beam generation location so that any positioning of the light source relative to the two interdigitated affinity gratings is no longer needed. In case the sensor comprises both the light source and the detector integrated in the planar waveguide or in the substrate, neither a positioning of the light source nor a positioning of the detector relative to the two interdigitated gratings is needed anymore. Rather, these components are already integrated into the sensor and are arranged at the proper location.

In other embodiments of the diffractometric sensor according to the invention, the resonant waveguiding structure arranged on the surface of the substrate comprises a metal layer, and the two interdigitated affinity gratings are arranged on a surface of the metal layer opposite to the surface of the metal layer facing the substrate. By way of example, the resonant waveguiding structure arranged on the surface of the substrate may be a single metal layer, which allows for the generation of a propagating surface plasmon at the surface of the metal layer opposite to the surface of the metal layer facing the substrate.

Here, the coherent light coupled into the metal layer propagates along the metal layer (i.e. it couples into a propagating surface plasmon), with an evanescent field thereof propagating along the surface of the metal layer opposite to the surface of the metal layer facing the substrate. On this surface of the metal layer opposite to the surface of the metal layer that faces the substrate, the two interdigitated affinity gratings are arranged.

In yet some further embodiments of the diffractometric sensor according to the invention, the affinity elements of the first type contained in the first unit cells of the first grating and the affinity elements of the second type contained in the second unit cells of the second grating are obtained using bioorthogonal coupling chemistries. The use of bioorthogonal coupling chemistries to obtain the affinity elements of the first type (contained in the unit cells of the first grating) and the affinity elements of the second type (contained in the unit cells of the second grating) allows one type of affinity elements—either the first type or the second type—to bind with biomolecules as target molecules while the respective other type of affinity elements—the second type or the first type—cannot bind with biomolecules as target molecules.

### BRIEF DESCRIPTION OF THE DRAWINGS

Further advantageous aspects and embodiments become apparent from the following description of aspects and embodiments of the invention with the aid of the schematic drawings in which:

FIG. 1 shows embodiments of the diffractometric sensor according to the invention in which the two interdigitated affinity gratings are arranged on the surface of a substrate;

FIG. 2 shows embodiments of the diffractometric sensor according to the invention in which a planar waveguide is arranged on the surface of a substrate, and in which the two interdigitated affinity gratings are arranged on the surface of the planar waveguide opposite to that surface of the planar waveguide facing the substrate;

FIG. 3 shows embodiments of the diffractometric sensor according to the invention in which a planar waveguide is arranged on the substrate and, with an optical coupler or an optical decoupler or both being arranged on the planar waveguide;

FIG. 4 shows one-dimensional, two-dimensional and three-dimensional embodiments of the first and second unit cells of the two interdigitated affinity gratings of the diffractometric sensor according to the invention;

FIG. 5 shows embodiments of the first and second unit cells of the two interdigitated affinity gratings, with identical or different affinity elements, and with or without scattering elements;

FIG. 6 shows embodiments of the first and second unit cells of the two interdigitated affinity gratings, with binding sites for scattering elements (and with or without such scattering elements being bound to these binding sites);

FIG. 7 shows a further embodiment of the first and second unit cells, with combined affinity elements/scattering elements and with separate affinity elements and scattering elements;

FIG. 8 shows further embodiments of the first and second unit cells, at different times during an assay, together with the respective scattering mass difference over time;

FIG. 9 shows further embodiments of the first and second unit cells, at different times during an assay, together with the respective change in intensity over time during the assay;

FIG. 10 shows further embodiments of the first and second unit cells, at different times during an assay, for detecting a specific target out of a group of targets that may bind to the affinity elements of one of the first and second unit cells, together with the respective scattering mass difference over time;

FIG. 11 shows further embodiments of the first and second unit cells, at different times during an assay, for detecting a specific amount of a target bound to the affinity elements, together with the respective intensity over time and together with the respective mass difference over time;

FIG. 12 shows an array comprising a plurality of diffractometric sensors according to the invention, and

FIG. 13 shows a signal representing the difference in scattering mass of the two interdigitated affinity gratings to illustrate the reaction kinetics during immobilization of the affinity elements in the first and second unit cells using bioorthogonal coupling chemistry.

### DESCRIPTION OF EMBODIMENTS

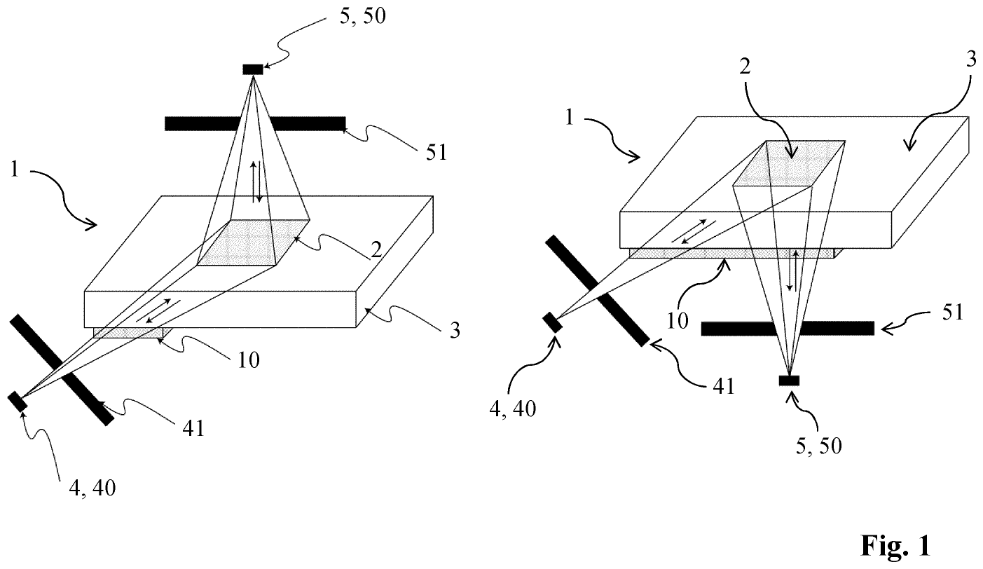

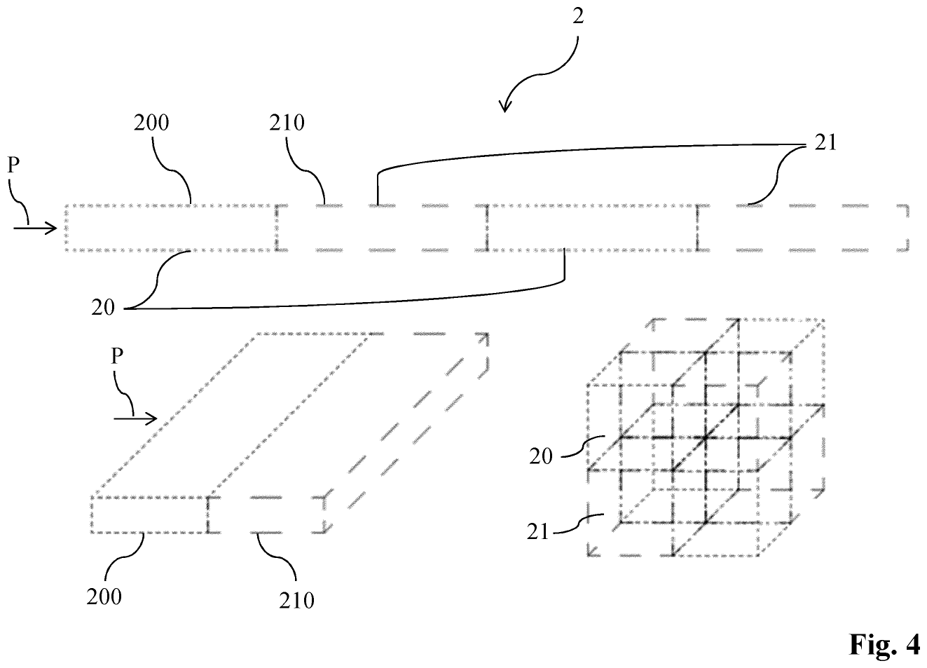

In FIG. 1 embodiments of a diffractometric sensor 1 according to the invention are shown. In the embodiments shown in FIG. 1 two interdigitated affinity gratings 2 (collectively referred to by reference sign 2, but comprising a first affinity grating 20 and a second affinity grating 21, see FIG. 4) are arranged on a surface of a substrate 3. On the left hand side in FIG. 1 an embodiment is shown where a light source 4 capable of generating a beam of coherent light of a predetermined wavelength (a monochromatic light source) is arranged at a predetermined beam generation location 40 beneath the diffractometric sensor 1, with a beam shaping aperture 41 being arranged in the optical path of the beam of coherent light from the light source 4 towards the diffractometric sensor 1. An optical coupler 10 is arranged on the lower surface of substrate 3 for coupling the beam of coherent light into the substrate 3 and directing the beam of coherent light to impinge on the interdigitated affinity gratings 2. A detector 5 is arranged at a predetermined detection location 50 above the substrate 3, with a beam shaping aperture 51 being arranged in the optical path of the beam of light diffracted by the two interdigitated affinity gratings 2. It goes without saying that the locations of the light source 4 and the detector 5 may be interchanged due to the optical path of the coherent light being reversible, this being illustrated by the two arrows in the respective portion of the beam of coherent light. It is further noteworthy that the predetermined beam generation location 40 (i.e. the location where the light source 4 is arranged) and the predetermined detection location 50 (i.e. the location where the detector 5 is arranged) as well as the wavelength of the beam of coherent light generated by the light source 4 must be known (see the above remarks with respect to a possible tuning range of the sensor), as the two interdigitated affinity gratings 2 are configured such that only for this specific combination of the predetermined beam generation location 40, the predetermined detection location 50 and the predetermined wavelength of the light source 4, the diffractometric sensor 1 is operable in the manner that will be explained further below.

The embodiment of the diffractometric sensor 1 shown on the right hand side in FIG. 1 is very similar to the embodiment shown on the left hand side in FIG. 1. The difference here is that the light source 4 and the detector 5 are both arranged beneath the substrate 3, and that the optical coupler 10 functions both as a coupler (for the beam of coherent light coming from the light source 4) and as a decoupler (for the beam of diffracted light coming from the two interdigitated affinity gratings 2) at the same time.

At first glance, the embodiments of the diffractometric sensor 1 shown in FIG. 1 appear to be well-known, however, the two interdigitated affinity gratings 2 (to be explained in more detail) make the diffractometric sensor 1 of this invention special and superior over prior art sensors.

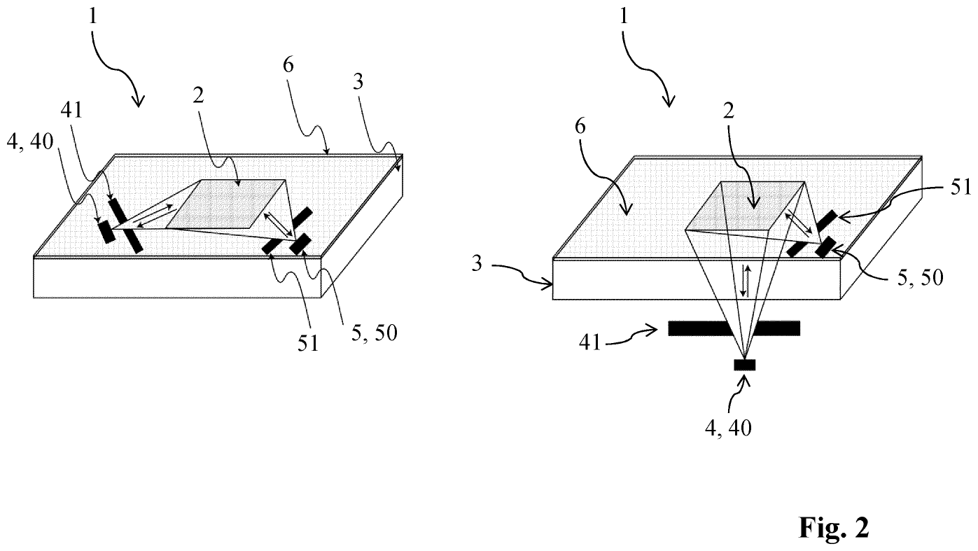

In FIG. 2 embodiments of the diffractometric sensor 1 according to the invention are shown in which a planar waveguide 6 is arranged on the surface of the substrate 3, and in which the two interdigitated affinity gratings 2 are arranged on the surface of the planar waveguide 6 opposite to that surface of the planar waveguide 6 facing the substrate 3. Further, in the embodiment shown on the left hand side in FIG. 2, the light source 4, aperture 41, detector 5 and aperture 51 are integrated in the diffractometric sensor 1, in that they are either arranged in the planar waveguide 6 or in the substrate 3. Thus, the embodiment shown on the left hand side in FIG. 1 represents a complete photonic integrated circuit. In the embodiment shown on the right hand side in FIG. 2, the light source 4 and the aperture 41 are not integrated in the diffractometric sensor 1 but are external to the diffractometric sensor 1, whereas the detector and the aperture 51 are integrated into the sensor.

Alternatively, in the embodiment shown on the right hand side in FIG. 2, the light source 4 and aperture 41 may be integrated into the sensor 1 whereas the detector 5 and aperture 51 may be arranged external to the sensor 1.

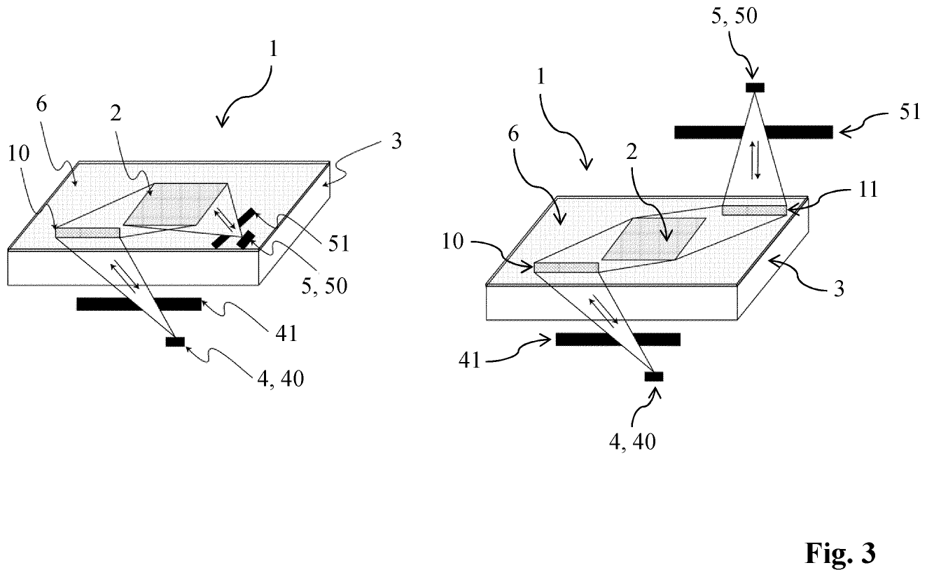

In FIG. 3 embodiments of the diffractometric sensor 1 according to the invention are shown in which again a planar waveguide 6 is arranged on the substrate 3. The embodiment of the diffractometric sensor 1 shown on the left hand side is very similar to the embodiment shown on the right hand side in FIG. 2, however, in addition an optical coupler 10 is arranged on the surface of the planar waveguide 6 and thus forms an integral part of the diffractometric sensor 1. The optical coupler 10 couples the coherent light coming from the light source into the planar waveguide 6 in a manner such that the coherent light propagates in the planar waveguide 6 to impinge on the two interdigitated affinity gratings 2. The coherent light diffracted by these two interdigitated affinity gratings 2 impinges (through aperture 51) onto the detector 5 which is integrated in the sensor 1 and which is arranged either in the planar waveguide 6 or in the substrate 3. Again, it is to be noted that it is also conceivable that instead of the light source 4 being external and the detector 5 being integrated in the sensor 1, the light source 4 may form an integral part of the sensor 1 while the detector 5 may be arranged external to the sensor 1.

The embodiment of the diffractometric sensor 1 shown on the right hand side in FIG. 3 differs from the embodiment shown on the left hand side in that both the light source 4 (and associated aperture 41) as well as the detector 5 (and associated aperture 51) are external to the diffractometric sensor 1 (i.e. none of them is integrated in the sensor 1). However, an optical coupler 10 as well as an optical decoupler 11 are arranged on the planar waveguide 6, for coupling the coherent light coming from the light source 4 (through aperture 41) into the planar waveguide 6 and directing it to impinge on the two interdigitated affinity gratings 2, and for decoupling the light diffracted by the two interdigitated affinity gratings 2 from the planar waveguide 6 an directing it (through aperture 51) to impinge on detector 5.

Thus, in the embodiment of the diffractometric sensor 1 shown on the right hand side in FIG. 3 the optical coupler 10 and the optical decoupler 11 are integrated in the diffractometric sensor 1.

With the aid of FIG. 1-FIG. 3 embodiments have been described how a diffractometric sensor 1 may look like from a more general constructional point of view. In the following, it is explained how the two interdigitated affinity gratings 2 may be embodied and how they work.

Generally, the two interdigitated gratings 2 may be embodied as one-dimensional interdigitated affinity gratings, two-dimensional interdigitated affinity gratings, or three-dimensional interdigitated gratings. As can be seen in FIG. 4, the two interdigitated affinity gratings 2 (collectively referred to in FIG. 1-FIG. 3), regardless of whether one-dimensional, two-dimensional or three-dimensional, generally comprise a first affinity grating 20 and a second affinity grating 21. The first affinity grating 20 comprises first unit cells 200 and the second affinity grating 21 comprises second unit cells 210. Each of the first unit cells 200 and the second unit cells 210 comprise affinity elements (to be discussed in more detail below). The first unit cells 200 comprise a first type of affinity elements and the second unit cells 210 comprise a second type of affinity elements. While in general the first type of affinity elements and the second type of affinity elements can be identical, preferably the first type of affinity elements and the second type of affinity elements are different.

In general, the dimensions of the first unit cells 200 and the second unit cells 210 (at least in the direction of propagation of the coherent light) are smaller than the predetermined wavelength of the coherent light. The direction of propagation of the coherent light is indicated by the arrow P in FIG. 4.

In the uppermost embodiment shown in FIG. 4, the first unit cells 200 (bounded by dotted lines) and the second unit cells 210 (bounded by dashed lines) are those of a one-dimensional first affinity grating 20 comprising the first unit cells 200 and those of a second one-dimensional affinity grating 21 comprising the second unit cells 210, with the direction of propagation being indicated by the arrow P. Such one-dimensional affinity gratings 20, 21 may be embodied as filament-like structures of alternatingly arranged first unit cells 200 and second unit cells 210 through which the coherent light propagates. Due to this filament-like structure the one-dimensional affinity gratings need not be geometrically arranged in the straight configuration shown in FIG. 4 but may be arranged in various other geometric arrangements so that the coherent light propagating through these unit cells may be guided in any desired direction, e.g. as this is the case in a photonic integrated circuit.

The coherent light of the predetermined wavelength diffracted by target molecules bound to the affinity elements or by scattering elements contained in the first unit cells 200 and the second unit cells 210 is diffracted to the same predetermined detection location 50 where the detector 5 (see FIG. 1-FIG. 3) is arranged, regardless of whether the first affinity grating 20 (comprising the unit cells 200) and the second affinity grating 21 (comprising the unit cells 210) are embodied as one-dimensional, two-dimensional, or three-dimensional affinity gratings.

In the lower embodiment shown on the left hand side in FIG. 4, the first unit cells 200 are those of a two-dimensional first affinity grating 20 and the second unit cells 210 are those of a two-dimensional second affinity grating 21. The direction of propagation of the coherent light is again illustrated by the arrow P.

Finally, in the lower embodiment shown on the right hand side in FIG. 4 the first unit cells 200 are those of a three-dimensional first affinity grating 20 and the unit cells 210 are those of a three-dimensional second affinity grating 21. In this embodiment, the Bragg-condition must be fulfilled for each of the first affinity grating 20 (comprising the first unit cells 200) and the second affinity grating 21 (comprising the second unit cells 210) when the light source 4 is arranged at the predetermined beam generation location 40 and the detector 5 is arranged at the predetermined detection location 50.

Regardless of whether one-dimensional, two-dimensional or three-dimensional, the coherent light diffracted by target molecules bound to the first type of affinity elements comprised by the first unit cells 200 (or by any scattering elements arranged in the first unit cells 200) is diffracted to the predetermined detection location 50 where detector 5 is arranged so as to constructively interfere at this predetermined detection location 50 with a first phase. The coherent light diffracted by target molecules bound to the second type of affinity elements comprised by the second unit cells 210 (or by any scattering elements arranged in the second unit cells 210) is also diffracted to the predetermined detection location 50 where the detector 5 is arranged so as to constructively interfere at this predetermined detection location 50 with a second phase.

However, this second phase is inverse to the first phase, so that the diffracted light having the first phase and the diffracted light having the second phase interfere at the predetermined detection location 50 where the detector 5 is arranged. Or to say it in other words, the two interdigitated gratings 20 and 21 form an optical comparator.

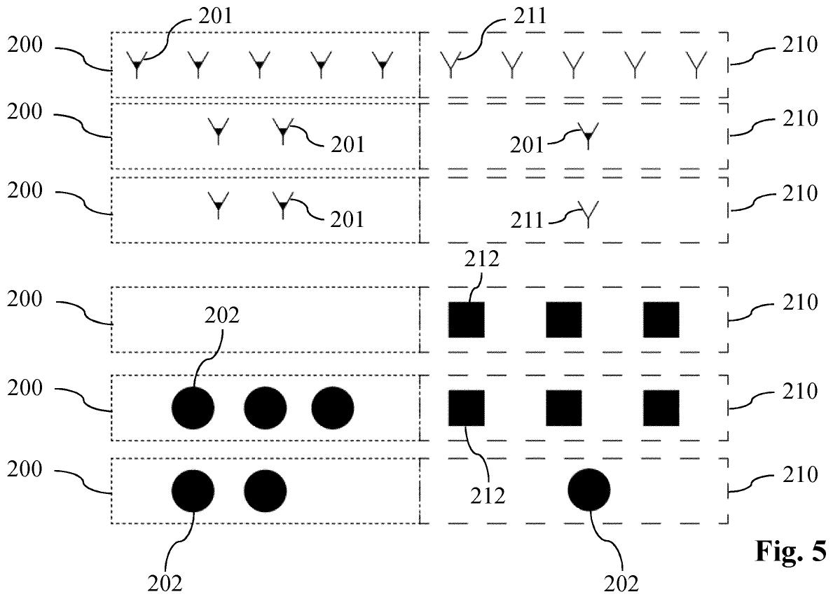

With reference now to FIG. 5, embodiments are discussed how the first unit cells 200 and the second unit cells 210 of the two interdigitated affinity gratings, i.e. of the first affinity grating 20 and the second affinity grating 21, may be embodied.

For the sake of simplification, only one first unit cell 200 of the first affinity grating 20 and one adjacently arranged second unit cell 210 of the second affinity grating 21 are shown in FIG. 5 and the subsequent figures. Again, the first unit cells 200 of the first affinity grating 20 are bounded by dotted lines while the second unit cells 210 of the second affinity grating 21 are bounded by dashed lines.

As can be seen in the uppermost embodiment shown in FIG. 5, a first type of affinity elements 201 (indicated by a Y-shape partially filled at the location where the two upper arms of the ‘Y’ meet) is arranged in the first unit cell 200, and a different second type of affinity elements 211 (indicated by a Y-shape, not filled) is arranged in the second unit cell 210. A first type of target molecules may bind only to the first type of affinity elements 201 while a second type of target molecules (different from the first type of target molecules) may bind only to the second type of affinity elements 211. A sample to be analyzed may be applied to the first unit cell 200 and the second unit cell 210 in order to detect whether the first type of target molecules or the second type of target molecules is contained in the sample. In case the first type of target molecules is contained in the sample (and consequently binds to the first type of affinity elements 201, thereby changing the scattering mass of the first unit cell 200), a signal is generated at the detection location having a first phase. In case the second type of target molecules is contained in the sample to be analyzed (and consequently binds to the second type of affinity elements 211, thereby changing the scattering mass of the second unit cell 210), a signal is generated at the detection location having a second phase (inverse to the first phase). The way how it may be detected whether the first type or the second type of target molecules is contained in the sample is described further below in connection with specific embodiments.

In the second embodiment from the top of FIG. 5, the same type of affinity elements 201 is arranged in the first unit cell 200 and in the second unit cell 210, however, with different concentrations (this being indicated by two affinity elements 201 of the first type being arranged in the first unit cell 200 while only one single affinity element 201 of the first type being arranged in the second unit cell 210). As a result, if target molecules of the first type are contained in the sample to be analyzed, two target molecules of the first type of target molecules bind to the two affinity elements 201 of the first type contained in the first unit cell 200 while only one target molecule of the first type of target molecules (same type) binds to the (single) affinity element 201 of the first type contained in second unit cell 210. Thus, the change in scattering mass of the first unit cell 200 is larger than the change in scattering mass of the second unit cell 210 and, consequently, a corresponding differential signal is generated at the detection location. Although not shown in the drawings, the same result can be achieved with different spatial arrangements of the same type of affinity element (e.g. the first type of affinity elements 201) in the first unit cell 200 and the second unit cell 210. That is to say, although the same amount of the first type of affinity elements 201 may be contained in the first unit cell and in the second unit cell, the spatial arrangement of the first type of affinity elements 201 in the first unit cell 200 and in the second unit cell may vary significantly: In the first unit cell 200 a plurality of the affinity elements 201 are arranged such that they may bind to only one target molecule of the first type while in the second unit cell each affinity elements 201 binds to one target molecule of the first type, so that in the end a different total number of target molecules of the first type are bound to the affinity elements 201 contained in the first unit cell 200 and the second unit cell 210.

In the third embodiment from the top of FIG. 5, it is illustrated that the first type of affinity elements 201 may be present in the first unit cell 200 while the second type of affinity elements 211 may be present in the second unit cell 210. In addition, different concentrations of the affinity elements and/or different spatial arrangements of the affinity elements may be present in the first unit cell 200 and the second unit cell 210.

In the third embodiment from the bottom of FIG. 5, it is illustrated that scattering elements (other than target molecules) may be provided in one of the unit cells. In this embodiment a second type of scattering elements 212 (indicated by filled squares) are arranged in the second unit cell 210. In the second lowermost embodiment shown in FIG. 5, a first type of scattering elements 202 is arranged in the first unit cell 200 (indicated by filled circles) while the second type of scattering elements 212 is arranged in the second unit cell 210. In the lowermost embodiment shown in FIG. 5, it is illustrated that only one type of scattering elements, here the first type of scattering elements 202, may be arranged in the first unit cell 200 as well as in the second unit cell 210, however, at different concentrations.

In the embodiments shown in FIG. 5, the first type of scattering elements 202 and the second type of scattering elements 212 are arranged in the unit cells without being removable (for example, they may be immobilized in the unit cells). The arrangement of scattering elements in the unit cells may be one way of providing a particular level of the bias signal at the detection location (which corresponds to a difference in scattering mass, see above).

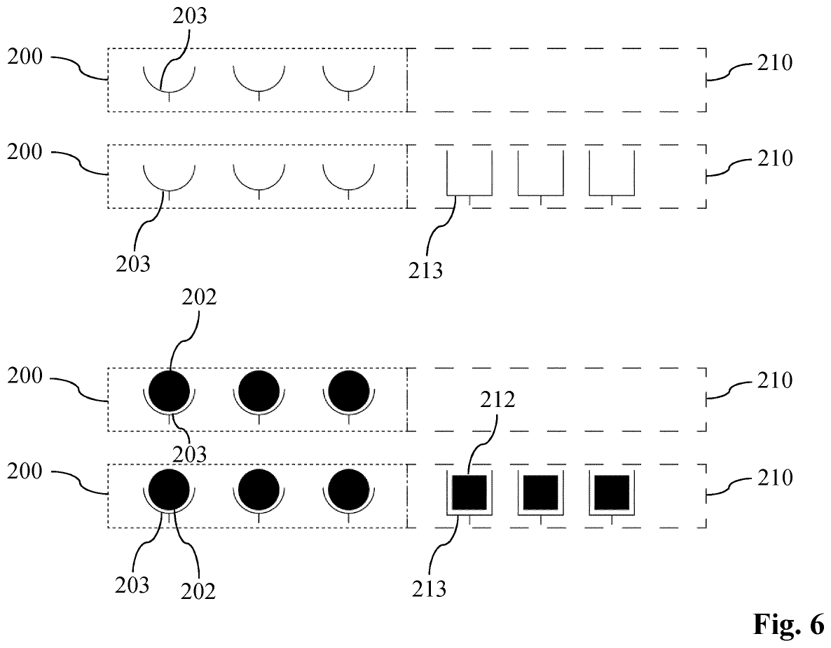

With reference now to FIG. 6, in the uppermost embodiment shown in FIG. 6, binding sites (indicated by cup-like elements of half-circle shape) which are capable of binding the first type of scattering elements 202 may be arranged in one of the first unit cell 200 and the second unit cell 210. In the embodiment shown, binding sites 203 of a first type are arranged in the first unit cell 200. Such binding sites 203 practically do not change the bias signal at the detector, but allow the first type of scattering elements 202 to be added to the first unit cell 200 and to be bound to the first type of binding sites 203 (as shown in the second lowermost embodiment shown in FIG. 6), and also allow the scattering elements 202 to be removed from the first unit cell 201 by being cleaved from the binding sites 203 should this become desirable. In the second embodiment from the top of FIG. 6, binding sites 203 of the first type which are capable of binding the first type of scattering elements 202 are arranged in the first unit cell 200, and binding sites 213 of a second type (indicated by cup-like elements of square-like shape with an open top) capable of binding the second type of scattering elements 212 are arranged in the second unit cell 210. This allows for adding the first type of scattering elements 202 and the second type of scattering elements 212 to the first unit cell 200 and the second unit cell 210 such that these bind to the corresponding first type of binding sites 203 and second type of binding sites 213 (as shown in the lowermost embodiment of FIG. 6). Also, this allows for later removal of the scattering elements (e.g. during an assay) from the unit cells by being cleaved from the binding sites.

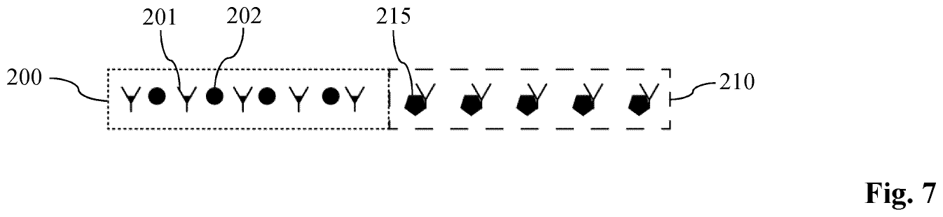

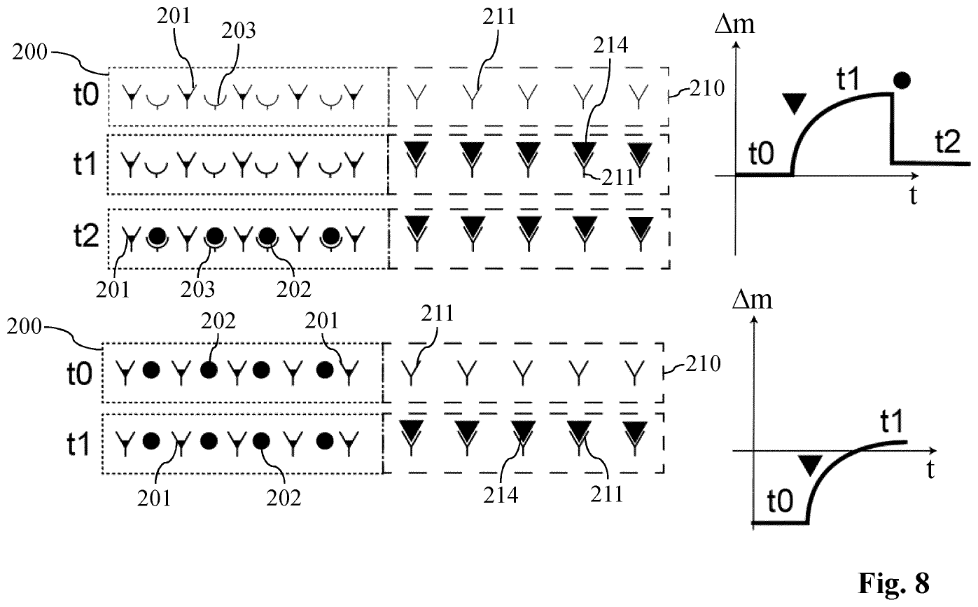

With reference now to FIG. 7, an embodiment is shown in which combined affinity elements/scattering elements 215 (indicated by a filled pentagon with an attached Y-shaped affinity element) are arranged in the second unit cell 210, whereas in the first unit cell 200 there are arranged separate affinity elements 201 and scattering elements 202. The separate scattering elements 202 are arranged in the first unit cell 200 to compensate for the scattering mass of the combined affinity elements/scattering elements 214 arranged in the second unit cell 210 in order to keep the bias generated by the sensor in the desired range. With reference to the embodiment shown at the top of FIG. 8, the state of the first unit cell 200 and the second unit cell 210 are shown during an assay, as well as a corresponding diagram showing the scattering mass difference Am (ordinate) over time t (abscissa) during the assay.

The first unit cell 200 contains the first type of affinity elements 201 as well as the first type of binding sites 203 capable of binding to the first type of scattering elements 202.

As mentioned already, the binding sites 203 practically do not generate any bias signal at the detector. The second unit cell 210 contains the second type of affinity elements 211. In this assay, it is known that the target molecules 214 of the second type are contained in the sample to be analyzed and will bind to the second type of affinity elements 211 contained in the second unit cell 210, and the amount of target molecules 214 (scattering mass) that will bind to the affinity elements 211 is also known.

During the time period t0, the initial state of the first unit cell 200 and the second unit cell 210 is shown. In the diagram showing the scattering mass difference over time (Am over t), this is represented by a scattering mass difference am which is practically zero so that a bias is generated at the detector 5 that corresponds to a scattering mass difference that is very small. As has been explained further above, in general the bias may correspond to a scattering mass difference of the first affinity grating 20 and the second affinity grating 21 which is in the range of 0.001 pg/mm² to 30000 pg/mm² (including all unit cells 200 of the first affinity grating and all unit cells 210 of the second affinity grating 21), and for many practical embodiments the bias may correspond to a scattering mass difference which is in the range of 0.1 pg/mm² to 1000 pg/mm², more particularly in the range of 0.1 pg/mm² to 100 pg/mm², and even more particularly in the range of 1 pg/mm² to 10 pg/mm².

Like in the above-discussed embodiments, in the following only one first unit cell 200 and one second unit cell 210 will be looked at.

During the time period t1, the target molecules 214 of the second type (indicated by filled triangles having downwardly pointing tips) are applied to the sensor 1, and in particular to the two interdigitated affinity gratings 2 of the sensor 1 (see FIG. 1-FIG. 3), i.e. to the first affinity grating 20 comprising the first unit cells 200 and the second affinity grating 21 comprising the second unit cells 210. As only the second type of affinity elements 211 (Y-shape, not filled) is capable of binding with the target molecules 214 of the second type (in this regard it is referred to the above remarks with respect to the capability of an affinity element to bind to only one type of target molecule), the target molecules 214 only bind to the second type of affinity elements 211 contained in the second unit cell 210, but do not bind to the first type of affinity elements 201 contained in the first unit cell 200. This binding of the target molecules 214 of the second type to the affinity elements 211 contained in the second unit cell 210 leads to an increase of the scattering mass difference am, as can be seen in the diagram.

At the end of time period t1 (or alternatively dynamically during the assay), scattering elements 202 of the first type (filled circles) are added which bind to the first type of binding sites 203 contained in the first unit cell 200. The amount of the added scattering elements 202 (scattering mass) is precisely known. Accordingly, after having added the scattering elements 202 the scattering mass difference Am (and thus the bias) is small again in the time period t2. The signal at the detector which is representative of the scattering mass difference Am can then be measured, and since the added amount (scattering mass) of the scattering elements 202 is precisely known, it is possible to determine the exact amount of the second type of target molecules 214 (scattering mass) that has actually bound to the second type of affinity elements 211 with great accuracy using inexpensive measurement equipment. It is obvious to the skilled person that the same functionality could also be achieved by having tunable scattering elements in unit cell 200 or 210 instead of having the binding sites 203 in the unit cells 200.

With reference now to the embodiment shown at the bottom of FIG. 8, again the state of the first unit cell 200 and the second unit cell 210 are shown during another assay, as well as a diagram showing the scattering mass difference am (ordinate) over time t (abscissa) during the assay.

The unit cell 200 again comprises affinity elements 201 of the first type, however, in this embodiment the first unit cell 200 additionally comprises a known amount (scattering mass) of the scattering elements 202 which may be immobilized in the first unit cell 200. The second unit cell 210 again contains the second type of affinity elements 211. In this assay, it is again known that the target molecules 214 of the second type will bind to the second type of affinity elements 211 contained in the second unit cell 210, and the amount of target molecules (scattering mass) that will bind to these affinity elements 211 is also known.

At the time t0, due to the presence of the scattering elements 202 in the first unit cell 200, the scattering mass difference am is large, as the target molecules 214 of the second type have not yet been applied to the sensor 1. Once the target molecules 214 of the second type are applied to the sensor they bind to the second type of affinity elements 211 contained in the second unit cell 210, so that the scattering mass difference am decreases until the scattering mass difference am is small again. The signal at the detector which is representative of the scattering mass difference am can then be measured, and since the amount (scattering mass) of the scattering elements 202 that are immobilized in the first unit cell 200 is precisely known, it is possible to determine the exact amount of the second type of target molecules 214 (scattering mass) that has actually bound to the second type of affinity elements 211 with great accuracy using inexpensive measurement equipment.

From the embodiments shown in FIG. 8, it can be seen that the scattering mass difference am in the embodiment shown at the top of FIG. 8 first has a positive sign (as the target molecules 214 bind to the affinity elements 211 of second unit cell 210 before the scattering mass compensation is performed by adding the scattering elements 202 to the first unit cell 200). In the embodiment shown at the bottom of FIG. 8, the scattering mass difference first has a negative sign (due to the scattering mass being larger in the first unit cell 200 before the scattering mass compensation is performed by the target molecules 214 binding to the affinity elements 211 of the second unit cell 210). In terms of the coherent light diffracted to the predetermined detection location 50 where the detector 5 is arranged, this means that in the embodiment shown at the top of FIG. 8 the coherent light diffracted by the target molecules 214 bound to the affinity elements 211 of the second unit cell constructively interferes at the detection location and has a second phase (before scattering mass compensation starts by adding the scattering elements 202 to the first unit cell 200).

In contrast thereto, in the embodiment shown at the bottom of FIG. 8 the coherent light diffracted by the scattering elements 202 of the first unit cell 200 constructively interferes at the detection location 50 where the detector 5 is arranged with a first phase that is inverse to the second phase (before scattering mass compensation starts by applying the target molecules 214 to the sensor and allowing them to bind to the affinity elements 211 of the second unit cell 210).

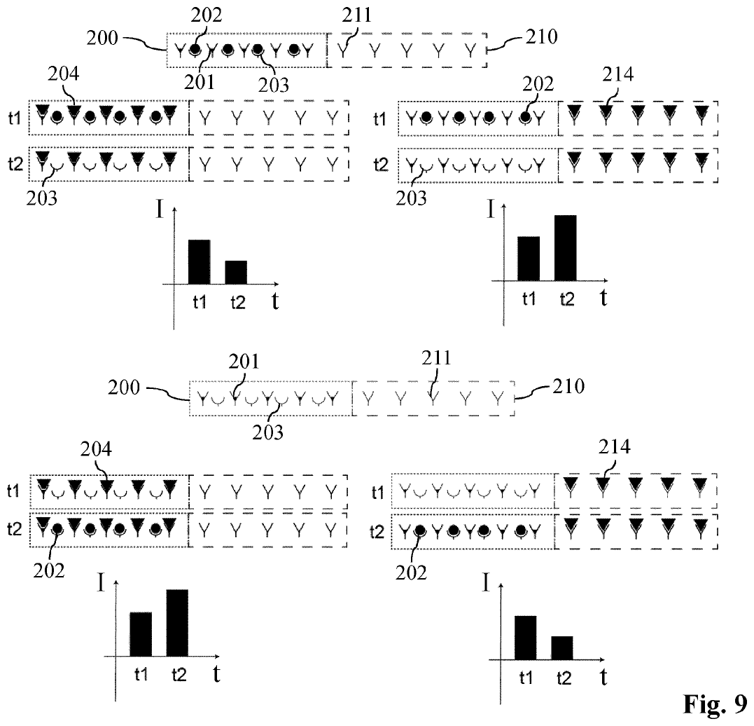

With reference to the embodiment shown at the top of FIG. 9, the state of the first unit cell 200 and the second unit cell 210 are shown at an initial state prior to starting an assay in which it is not known whether the target molecules contained in a sample to be analyzed bind to the first type of affinity elements 201 contained in the first unit cell 200 or to the second type of affinity elements 211 contained in the second unit cell 210.

Detection of whether the target molecules bind to the first unit cell 200 or to the second unit cell 210 is possible here by monitoring the change in intensity I detected at the predetermined detection location 50 where the detector 5 is arranged at different points in time t1 and t2. For that purpose, in the first unit cell 200 the first type of affinity elements 201 is arranged as well as a plurality of binding sites 203 to which a known amount (scattering mass) of scattering elements 202 has bound, which are cleavable from the binding sites 203.

Now the target molecules are added to the sensor, and it is to be determined whether these target molecules bind to the first type of affinity elements 201 contained in the first unit cell 200 or to the second type affinity elements 211 contained in the second unit cell 210.

Following the branch on the left hand side of the embodiment shown at the top of FIG. 9, the target molecules contained in the sample to be analyzed are target molecules 204 of a first type (triangles with truncated tip pointing downwardly) and bind to the first type of affinity elements 201 contained in the first unit cell 200. This results in a first intensity I being measured at the detector at the time t1, as can be seen in the diagram (intensity I—ordinate; time t—abscissa) below the branch on the left hand side. This first intensity I of the signal at the detector is caused by the difference in scattering mass of the first unit cell 200 (scattering mass of the scattering elements 202 plus scattering mass of the target molecules 204 of the first type bound to the affinity elements 201) and the second unit cell 210.

Next, the scattering elements 202 are cleaved and removed from the first unit cell 200. This leads to a decrease in the scattering mass difference, and thus to a decrease of the intensity I measured at the detector at the time t2. From this change (decrease) in intensity I it can be detected that the target molecules contained in the sample must be target molecules 204 of the first type that have bound to the first type of affinity elements 201 contained in the first unit cell 200.

Following the branch on the right hand side of the embodiment at the top of FIG. 9, the target molecules contained in the sample to be analyzed are target molecules 214 of the second type (triangles with tip pointing downwardly) and bind to the second type of affinity elements 211 contained in the second unit cell 210. This results in a first intensity I being measured at the detector at the time t1, as can be seen in the diagram (intensity I—ordinate; time t—abscissa) below the branch on the right hand side. This first intensity I of the signal at the detector is caused by the difference in scattering mass of the first unit cell 200 (scattering elements 202) and the second unit cell 210 (scattering mass of the target molecules 214 bound to the affinity elements 211).

Next, the scattering elements 202 are cleaved and removed from the first unit cell 200. This leads to an increase in the scattering mass difference between the first unit cell 200 and the second unit cell 210, and thus to an increase of the intensity I measured at the detector at the time t2. From this change (increase) in intensity I it can be detected that the target molecules contained in the sample must be target molecules 214 of the second type that have bound to the second type of affinity elements 211 contained in the second unit cell 210.

With reference to the embodiment shown at the bottom of FIG. 9, the first unit cell 200 and the second unit cell 210 are shown at an initial state prior to starting an assay in which it is not known whether the target molecules contained in a sample to be analyzed bind to the first type of affinity elements 201 contained in the first unit cell 200 or to the second type of affinity elements 211 contained in the second unit cell 210.

Whether the target molecules bind to the first unit cell 200 or to the second unit cell 210 can be detected by monitoring the change in intensity I detected at the predetermined detection location 50 where the detector 5 is arranged at different points in time t1 and t2. In contrast to the embodiment shown at the top of FIG. 9, however, no scattering elements 202 are arranged in the first unit cell 200, only the binding sites 203 capable of binding such scattering elements 202 are arranged in the first unit cell 200. Now the target molecules of the sample are added to the sensor, and it is to be determined whether these target molecules bind to the first type of affinity elements 201 contained in the first unit cell 200 or to the second type of affinity elements 211 contained in the second unit cell 210.

Following the branch on the left hand side of the embodiment at the bottom of FIG. 9, the target molecules contained in the sample to be analyzed are again target molecules 204 of a first type (triangles with truncated tip pointing downwardly) and bind to the first type of affinity elements 201 contained in the first unit cell 200. This results in a first intensity I being measured at the detector at the time t1, as can be seen in the diagram (intensity I—ordinate; time t—abscissa) below the branch on the left hand side. This first intensity I of the signal at the detector is caused by the difference in scattering mass of the first unit cell 200 (scattering mass of the target molecules 204 of the first type bound to the affinity elements 201) and the second unit cell 210.

Next, scattering elements 202 are added to the first unit cell 200. This leads to an increase in the scattering mass difference of the first unit cell 200 and the second unit cell 210, and thus to an increase of the intensity I measured at the detector at the time t2. From this change (increase) in intensity I it can be detected that the target molecules contained in the sample must be target molecules 204 of the first type that have bound to the first type of affinity elements 201 contained in the first unit cell 200.

Following the branch on the right hand side of the embodiment at the bottom of FIG. 9, the target molecules contained in the sample to be analyzed are target molecules 214 of the second type (triangles with tip pointing downwardly) and bind to the second type of affinity elements 211 contained in the second unit cell 210. This results in a first intensity I being measured at the detector at the time t1, as can be seen in the diagram (intensity I—ordinate; time t—abscissa) below the branch on the right hand side. This first intensity I of the signal at the detector is caused by the difference in scattering mass of the first unit cell 200 and the second unit cell 210 (scattering mass of the target molecules 214 bound to the affinity elements 211).

Next, the scattering elements 202 are added to the first unit cell 200. This leads to a decrease in the scattering mass difference of the first unit cell 200 and the second unit cell 210, and thus to a decrease of the intensity I measured at the detector at the time t2. From this change (decrease) in intensity I it can be detected that the target molecules contained in the sample must be target molecules 214 of the second type that have bound to the second type of affinity elements 211 contained in the second unit cell 210.

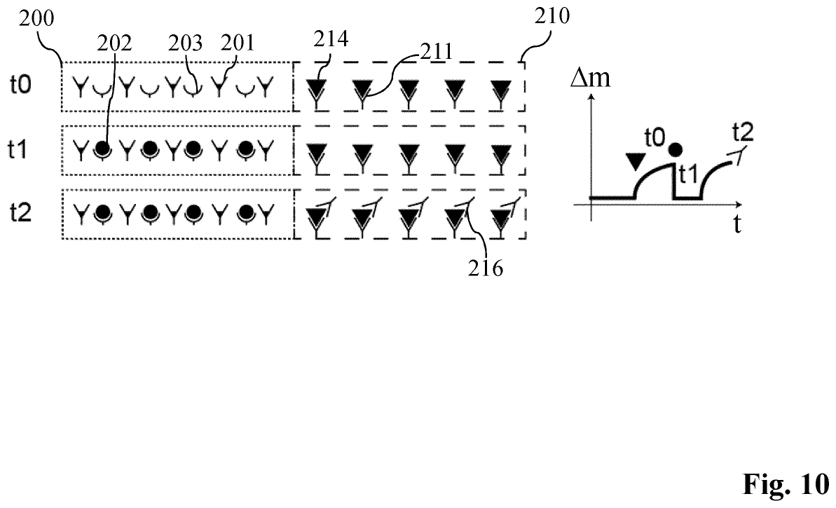

With reference to the embodiment shown in FIG. 10, an assay is shown in which initially the first unit cell 200 comprises the first type of affinity elements 201 as well as the binding sites 203 capable of binding the scattering elements 202 (however, with no scattering elements 202 being bound to the binding sites 203 yet). The second unit cell 210 initially comprises only the second type of affinity elements 211 (with no target molecules being bound yet, this initial state not being shown in FIG. 10). During the time period t0 the sample containing target molecules 214 of the second type (triangles with tip pointing downwardly) is applied to the sensor, and the target molecules 214 of the second type bind to the second type of affinity elements 211. This leads to an increase in the scattering mass difference am of the first unit cell 200 and the second unit cell 210, as can be seen from the diagram on the right hand side showing the mass difference Am over time t (Am—ordinate; t—abscissa). At the end of the period t0, the scattering elements 202 are added and bind to the binding sites 203 in the first unit cell 200. As a consequence of this scattering mass compensation, during the period t1 the scattering mass difference am is small again, as can be seen from the diagram on the right hand side. In case the second type of affinity elements 211 is capable of binding a certain type of target molecules 214 (e.g. a group of different target molecules 214 having a common binding site or moiety so that all target molecules 214 of this group may bind to the second type of affinity elements 211), it may be necessary to identify the specific target molecule of this type (or group). For example, at the end of time period t1, a specific type of detection antibody 216 may be added which is capable of binding only to one specific target molecule of the second type (or group) of target molecules 214. Thus, it is possible to identify the specific target molecule out of a type (or group) of target molecules 214 that may all bind to the second type of affinity elements 211. As the specific type of detection antibody 216 binds to the target molecules 214, the scattering mass difference am increases again during the time period t2, as can be seen from the diagram on the right hand side. Such a sensor is capable of detecting a target molecule in a sample that contains a large amount and a vast diversity of background molecules in a wash-free format.

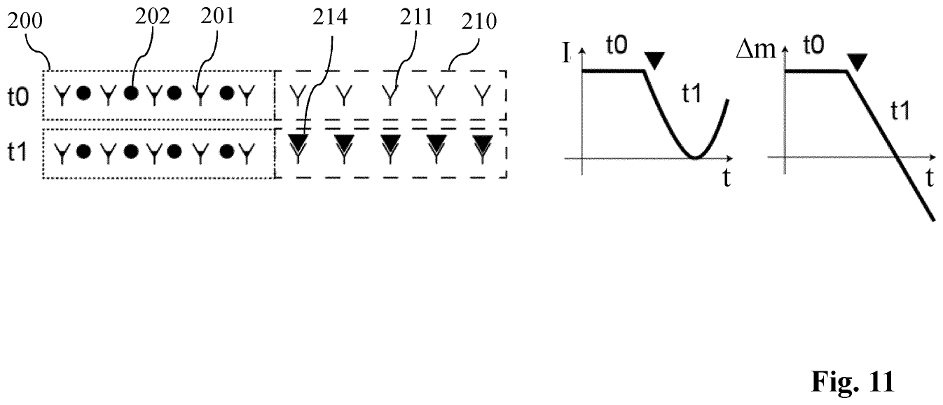

With reference to the embodiment shown in FIG. 11, an assay is shown in which a bias is selected allowing for an increase of the dynamic range of the measurement while keeping the accuracy required for the measurement low (so that the measurement can be performed using simple and inexpensive measurement equipment).

For that purpose, in the initial state at time period t0, the first unit cell 200 comprises the first type of affinity elements 201 as well as scattering elements 202 which are arranged in the first unit cell 200 (e.g. the scattering elements 202 are immobilized in the first unit cell 200). During this time period t0, the second unit cell 210 only comprises the second type of affinity elements 211, however, with no target molecules being bound thereto.

The two diagrams shown on the right hand side in FIG. 11 show the scattering mass difference am over time t (outermost right: scattering mass difference Am—ordinate; time t—abscissa) and the intensity I over time t (second outermost right: intensity I—ordinate; time t—abscissa). During the time period t0, the scattering mass difference am is positive (the scattering mass of the first unit cell 200 is larger than the scattering mass of the second unit cell 210 due to the scattering elements 202 contained in the first unit cell 200).

At the end of time period t0, a sample containing the second type of target molecules 214 is applied to the sensor, and the target molecules 214 of this second type start binding to the second type of affinity elements 211 contained in the second unit cell 210.

Glancing at the curve in the diagram showing the course of the scattering mass difference am over time t, it can be seen that —by way of example—the scattering mass difference am linearly decreases, and after a certain time during time period t1 (for example, at the time three of the target molecules 214 have bound to three affinity elements 211 contained in the second unit cell 210) the scattering mass difference Am is zero (the curve intersects the abscissa). At the predetermined detection location 50 where the detector 5 is located, the amplitude of the coherent light diffracted by the first unit cell 200 (representing the first affinity grating 20) and the second unit cell 210 (representing the second affinity grating 21) is directly proportional to the scattering mass difference am of the first unit cell 200 (representing the first affinity grating 20) and the second unit cell (representing the second affinity grating 21), due to the coherent light diffracted by the first unit cell 200 (representing the first affinity grating 20) and the coherent light diffracted by the second unit cell 210 (representing the second affinity grating 21) being inverse in phase, as has been explained above.

Glancing at the curve in the diagram showing the intensity I over time t, it can be seen that during the same time the intensity I also decreases to zero. The intensity I is proportional to the square of the amplitude of the coherent light at the predetermined detection location 50 where the detector 5 is located. This is why the curve of the intensity I over time has the shape of a parabola and decreases to zero following the curve of the parabola.

Glancing again at the curve in the diagram showing the scattering mass difference Am over time t, it can be seen that at the time all five target molecules 214 have bound to the five affinity elements 211 contained in second unit cell 210 the sign of the scattering mass difference am is negative (and so is the amplitude of the diffracted coherent light at the predetermined detection location 50 where the detector is arranged), due to the scattering mass of the second unit cell 210 (representing the second affinity grating 21) now being larger than the scattering mass of the first unit cell 200 (representing the first affinity grating 20).

When glancing at the curve in the diagram showing the intensity I over time t, it can be seen that during this time the intensity I increases again (due to the intensity I being proportional to the square of the scattering mass difference am or the amplitude of the diffracted coherent light).

Accordingly, providing a bias at the detector (here: by providing the scattering elements 202 in the first unit cell 200) may increase the dynamic range of measurement of the mass of target molecules while maintaining the requirements for the measurement accuracy and thus allow for using simple and inexpensive measurement equipment. Thanks to the continuous measurement, the sign of the mass difference am is known at all times.

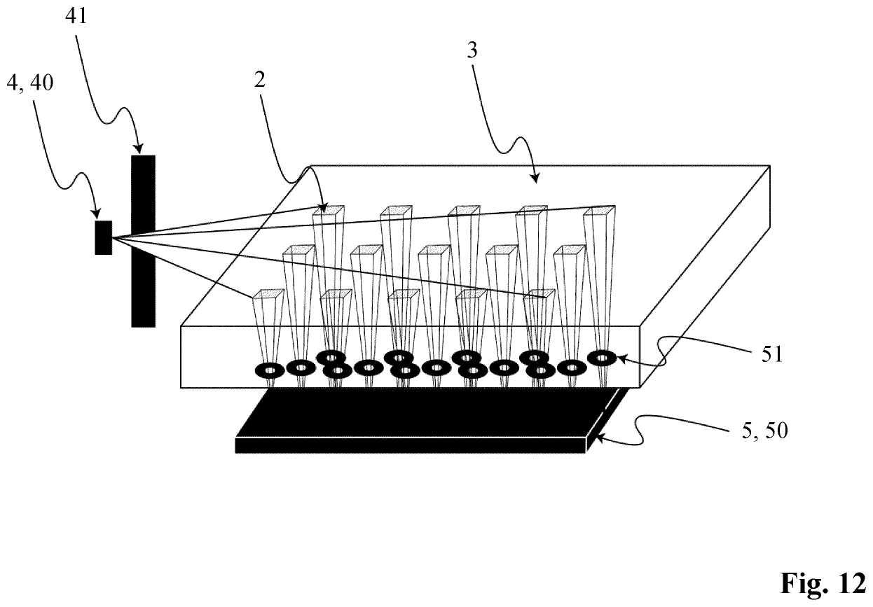

Finally, in FIG. 12 an embodiment of a sensor array is shown that comprises a plurality of individual diffractometric sensors according to the invention, each sensor comprising two interdigitated gratings 2. The substrates of the individual sensors may be formed by a common substrate 3. Further, a common light source 4 for generating a beam of coherent light of a predetermined wavelength is provided at a predetermined beam generation location 40, as well as a common aperture 41 allowing the coherent light to pass onto each of the interdigitated gratings 2 which diffract the coherent light of the predetermined wavelength to a common detector 5 (e.g. a CCD-array), with individual apertures 51 being provided for the light diffracted by the individual interdigitated gratings 2. In one embodiment of the sensor array, the individual respective interdigitated gratings 2 may all contain the same type of affinity elements but are differently biased (scattering mass difference) in order to allow for a maximum resolution of the measurement for a wide range of detectable scattering masses. In another embodiment of the sensor array, different interdigitated gratings 2 may comprise different affinity elements so that in case a sample is to be analyzed a characteristic ‘fingerprint’ of the sample can be determined using such sensor array (i.e. various different target molecules contained in a sample may be identified as being contained in the sample using the various different interdigitated gratings 2 in the manner described above). In yet other embodiments of the sensor array, highly specific sensors (which may only bind to one specific target molecule) may be arrayed with highly unspecific sensors (which may only detect a type/group of molecules or a specific feature or property of molecules, for example the hydrophilicity or hydrophobicity.

While the general teaching underlying the diffractometric sensor according to the invention has been explained above, some specific technical options for some elements of the sensor according to the invention will be explained in the following by way of example.

Composition of the Affinity Gratings or Unit Cells, Arrangement of Affinity Elements and Scattering Elements

In some embodiments, the first and second affinity gratings (unit cells) may only comprise the affinity elements. In addition, one of the first and second affinity gratings may comprise scattering elements. In other embodiments, the first affinity grating and the second affinity grating both may contain scattering elements of different types or number.

In some embodiments the unit cells may comprise a bulk material/framework material that can be any material that allows for diffusional entrance of the analyte or a group of analytes.

For example, the bulk material/framework material may comprise a polymer, preferably a non-fouling polymer, that can be functionalized with affinity elements or scattering elements. A mesh size/porosity of the bulk material/framework material can be adjusted to match the desired application.

In some embodiments, the unit cells of the gratings may contain a substrate that may be coated with a thin polymer (only a fraction of the height of the unit cells). In such embodiments the scattering elements can be arranged within the substrate in any configuration, but preferably in the ones described above).

Fabrication of the Scattering Elements

In some embodiments, before filling the unit cells with the bulk material/framework material the scattering elements may be formed by deposition of a dielectric material on the substrate or by etching the substrate. In other embodiments, during the formation of the bulk material/framework material in the unit cells, different polymerization times, crosslinking densities, thicknesses, porosities, etc. may be used to form the scattering elements. In yet other embodiments, after the bulk material/framework material of the unit cells has been formed, the scattering elements may be formed with the aid of light-induced precipitation or covalent immobilization of nanoparticles or large molecules using suitable crosslinking chemistries.

In some embodiments, only binding sites capable of binding scattering elements may be present in the unit cells. The binding sites of the different gratings may be capable of binding different types of scattering elements. The scattering elements may be cleavable from the binding sites so that they can be removed from the unit cells. In other embodiments the affinity elements themselves may be embodied as combined scattering elements/affinity elements.

Control of the Scattering Power/Scattering Strength of the Scattering Elements

The scattering elements can be inert/static or can be functional/tunable. ‘Inert/static’ means that the scattering power/scattering strength is fixed and cannot be changed by the operator or through experimental conditions. ‘Functional/tunable’ means that the scattering power/scattering strength can be adjusted by physical, chemical or biological measures. For example, ‘physical measures’ include the application of an external field to cause a change in refractive index in case the scattering elements are made from an electrooptic material, or magnetic pull-down in embodiments including an evanescent field. ‘Chemical measures’ include etching of the scattering elements, polymerization of the scattering elements, precipitation of the scattering elements at nucleation sites, redox reactions at the scattering elements, etc. ‘Biological measures’ include the binding of additional scattering mass to the scattering elements or enzymatic degradation of the scattering elements.

Illumination of the diffractometric sensor Preferably, the diffractometric sensor is illuminated with coherent light by means of an evanescent wave (e.g. using a planar waveguide) in order to reduce parasitic straylight, although illumination by freely-propagating beams is possible as well. The coherent light is preferably polarized, although this is not a requirement. The source may be tunable, either in wavelength (within a small range about the predetermined wavelength) or in the spatial direction in order to scan the diffraction condition. Array detectors may preferably be used in order to compensate for minor mechanical movement as well as for tuning of the diffraction condition. Preferably, apertures are used that can be static (chromium screen) or dynamic (LCD crystal displays).

Other Assay Formats

Any of the above mentioned sensor embodiments and applications of a controlled bias can be implemented with all known surface based assay formats, e.g. direct binding assays, competitive assays, enzymatic degradation assays, sandwich-assays, reverse phase assays, labeled assays, where the label comprises a scattering element. Of particular importance are assays where the affinity element comprises a recognition moiety that, upon interaction with the target, leads to cleavage of a scattering element. Such target-mediated cleavage reactions include, for example, CRISPR/Cas9 or similar detection systems. The affinity element comprising the recognition moiety may be linked to a scattering element that is released upon cleavage. In such case, scattering elements in the unit cells of the other grating may compensate for the scattering element that is linked to the recognition moiety.

Embodiments of the invention have been explained above, however, the invention is not limited to these embodiments. Rather, many changes and modifications can be made without departing from the teaching underlying the invention. Therefore, the scope of protection is defined by the appended claims.

Scattering Mass Tuning Based on Bioorthogonal Chemistries with Different Rate Constants

In this embodiment an affinity element 201 of a first type is immobilized in the first unit cells 200 of the first grating 20 (see, for example, FIG. 4 and FIG. 5) such that the coherent light of the predetermined wavelength interferes at the detection location 50 (see FIG. 1-FIG. 3) having the first phase, and an affinity element 211 of a second type (different from the first type) is immobilized in the second units cells 210 of the second grating 21 such that the coherent light of the predetermined wavelength interferes at the detection location 50 having the second phase inverse to the first phase.

To achieve this, the first unit cells 200 of the first grating 20 contain elements having a functional group that reacts with a certain kinetics with a particular functional group of the affinity element 201 of the first type (i.e. a macromolecule) to be immobilized in the first unit cells 200. The second unit cells 210 of the second grating 21 contain elements having a different functional group that also reacts with a different kinetics with the particular functional group of the affinity element 211 of the second type (i.e. a macromolecule), the rate (speed) of the reaction kinetics in the second unit cells 210 being preferentially by a factor two to ten different from the rate (speed) of the reaction kinetics in the first unit cells 200.

An exemplary embodiment of two biorthogonal coupling chemistries with different rates (speeds) of the reaction kinetics are (a) tetrazine reacting with transcyclooctene, and (b) methyltetrazine reacting with transcyclooctene

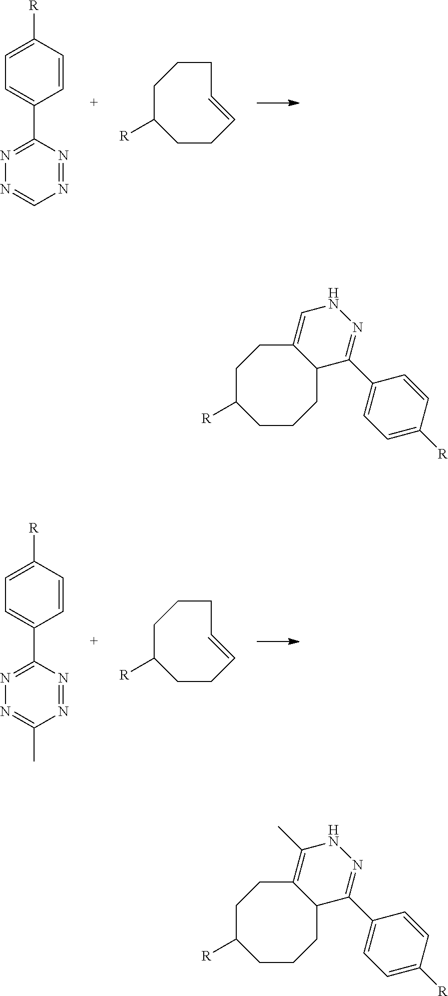

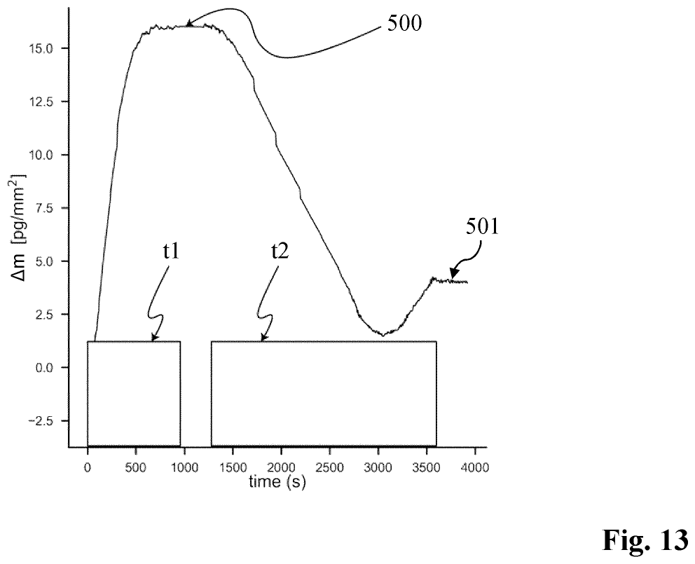

The kinetics of the bioorthogonal reactions (shown above) of tetrazine with transcyclooctene (the particular functional group of the (modified) affinity element of the first type to be immobilized in the first unit cells) and methyltetrazine with transcyclooctene (the particular functional group of the (modified) affinity element of the second type to be immobilized in the second unit cells) are described in the following with the aid of FIG. 13.

The reaction of tetrazine with transcyclooctene is roughly four to five times faster than the reaction of methyltetrazine with transcyclooctene. In principle, the difference in reaction speed can be adjusted by measures such as pH, solvent, ionic strength, temperature, acoustics and illumination. Accordingly, in a first step the affinity element 201 of the first type to be immobilized in the first unit cells 200 and having transcyclooctene as the functional group is applied to the surface of the diffractometric sensor 1 at the location of the interdigitated gratings 2. Due to the reaction speed of transcyclooctene with tetrazine (the functional group of the elements present in the first unit cells 200) being very considerably faster than the reaction speed of cyclooctene with methyltetrazine (the functional group of the elements present in the second unit cells 210) the transcyclooctene of the affinity element 201 of the first type binds to the tetrazine and thereby immobilizes the affinity elements 201 of the first type in the first unit cells 20 (while at the same time only very few affinity elements 201 of the first type are immobilized in the second unit cells 210 due to the very considerably lower reaction speed of the transcyclooctene with methyltetrazine). Incubation is performed at a suitable concentration, preferentially 1 μM (M=Molar=mol/l) of the transcyclooctene modified molecular moiety of the affinity element of the first type to be immobilized in the first unit cells of the first grating until the signal at the detector 5 preferentially forms a plateau 500 (FIG. 13). The time t1 needed for this incubation is shown in FIG. 13. The plateau 500 of the signal at the detector 5 indicates that most of the tetrazine has reacted with the transcyclooctene of the affinity elements 201 of the first type in the first unit cells 200.

Thereafter, the remaining affinity elements of the first type are removed from the surface of the diffractometric sensor 1 and the affinity elements 211 of the second type to be immobilized in the second unit cells 210 are applied to the surface of the diffractometric sensor 1 at the location of the interdigitated gratings 2, also having transcyclooctene as the functional group. Since most of the methyltetrazine present in the second unit cells 210 has not reacted with the transcyclooctene of the affinity elements 201 of the first type (due to the reaction speed being very considerably slower), this methyltetrazine is now allowed to react with the transcyclooctene of the affinity elements 211 of the second type. For that reason, a second incubation with a transcyclooctene modified molecular moiety of the affinity elements 211 of the second type to be immobilized in the second unit cells 210 of the second grating 21 is now performed preferentially until the signal at the detector 5 reaches a level 501 representing the bias that corresponds to the desired scattering mass difference Am. The time t2 needed for this second incubation is shown in FIG. 13. As can also be seen in FIG. 13, the level 501 of the signal at the detector 5 corresponds to a scattering mass difference Am of about 5 pg/mm².

The afore-described reaction pair is especially favorable due to its comparatively fast reaction speed (typically in the range of less than two hours) and its good compatibility with proteins as compared to other biorthogonal coupling chemistries, such as copper catalyzed click chemistry (CuAAC) which is only poorly performing with proteins and strain promoted click chemistry (SPAAC) which is comparatively slow in reaction speed (typically in the range of ten to twenty hours).

While embodiments of the invention have been described with the aid of the drawings and specific examples, the invention is not limited to these embodiments and examples, since various changes and modifications are conceivable without departing from the teaching underlying the invention. Rather, the scope of protections is defined by the appended claims.

1. Diffractometric sensor (1), comprising:
a substrate (3);
two interdigitated affinity gratings (2; 20, 21) arranged on the substrate, a first affinity grating (20) and a second affinity grating (21), the first affinity grating (20) comprising first unit cells (200) and the second affinity grating (21) comprising second unit cells (210),
the first unit cells (200) of the first affinity grating (20) comprising affinity elements (201) of a first type capable of binding with target molecules (204) of a first type,
and the second unit cells (210) of the second affinity grating (21) comprising affinity elements (211) of a second type capable of binding with target molecules (214) of a second type, wherein the first unit cells (200) of the first affinity grating (20) are configured and arranged such that coherent light of a predetermined wavelength generated at a predetermined beam generation location (40) and diffracted by target molecules (204) of the first type bound to the affinity elements (201) of the first type constructively interferes at a predetermined detection location (50) with a first phase,
wherein the second unit cells (210) of the second affinity grating are (20) configured and arranged such that the coherent light of the predetermined wavelength generated at the predetermined beam generation location (40) and diffracted by target molecules (214) of the second type bound to the affinity elements (211) of the second type constructively interferes at the predetermined detection location (50) with a second phase inverse to the first phase,
and wherein the first and second affinity gratings (20, 21) are balanced with respect to a scattering mass of the first and second affinity gratings (20, 21) to generate a bias signal at the predetermined detection location (50) that corresponds to a difference (Am) in the scattering mass of the first and second affinity gratings (20, 21) which is in the range of 0.001 pg/mm2 to 30000 pg/mm2.

2. Diffractometric sensor according to claim 1, wherein the bias signal corresponding to the difference (Am) in the scattering mass of the first and second affinity gratings (20, 21) is in the range of 0.1 pg/mm2 to 1000 pg/mm2, more particularly in the range of 0.1 pg/mm2 to 100 pg/mm2, and even more particularly in the range of 1 pg/mm2 to 10 pg/mm2.

3. Diffractometric sensor according to claim 1 wherein the concentration or the spatial arrangement of the affinity elements (201) of the first type in the first unit cells (200) and the concentration or the spatial arrangement of the affinity elements (211) of the second type in the second unit cells (210) are different.

4. Diffractometric sensor according to claim 3, wherein the affinity elements (201) of the first type and the affinity elements (211) of the second type are either identical or different.

5. (canceled)

6. Diffractometric sensor according to claim 1 wherein the affinity elements (201) of the first type are non-binding for the target molecules of the second type or the affinity elements (211) of the second type are non-binding for the target molecules of the first type, or both.

7. Diffractometric sensor according to claim 1, wherein at least one (20, 21) of the two interdigitated affinity gratings (2) further comprises binding sites (203, 213) capable of binding scattering elements (202, 212), wherein the at least one of the two interdigitated affinity gratings further comprises scattering elements (202, 212), and wherein the scattering elements (202, 212) are bound to the binding sites (203, 213).

8. (canceled)

9. Diffractometric sensor according to claim 7, wherein the scattering elements (202, 212) are arranged either in the first unit cells (200) or in the second unit cells (210) or in both the first and second unit cells (200, 210) of the two interdigitated affinity gratings.

10. (canceled)

11. Diffractometric sensor according to claim 7 wherein the scattering elements (202, 212) are tunable or cleavable to allow for adjustment of the scattering power or removal of the scattering elements (202, 212).

12. Diffractometric sensor according to claim 1, wherein the two interdigitated affinity gratings (2) are arranged on a surface of the substrate (3).

13. Diffractometric sensor according to claim 12, further comprising an optical coupler (10) configured and arranged to direct the coherent light coming from the predetermined beam generation location (40) to the two interdigitated affinity gratings (2) arranged on the surface of the substrate (3).

14. Diffractometric sensor according to claim 12, further comprising an optical decoupler (10) configured and arranged to direct the coherent light diffracted by the two interdigitated affinity gratings (2) to the predetermined detection location (50).

15. Diffractometric sensor according to claim 1 further comprising a resonant waveguiding structure arranged on the surface of the substrate (3), the resonant structure being configured to allow for coupling of the coherent light of the predetermined wavelength generated at the predetermined beam generation location (40) into the resonant waveguiding structure to generate an evanescent field propagating along an outermost surface of the resonant waveguiding structure opposite to a surface of the resonant waveguiding structure facing the substrate (3), and wherein the two interdigitated affinity gratings (2) are arranged on the outermost surface of the resonant waveguiding structure.

16. Diffractometric sensor according to claim 15, wherein the resonant waveguiding structure arranged on the surface of the substrate is a planar waveguide (6), and wherein the two interdigitated affinity gratings (2) are arranged on a surface of the planar waveguide (6) opposite to a surface of the planar waveguide (6) facing the substrate (3).

17. Diffractometric sensor according to claim 16, wherein the planar waveguide (6) is structured so as to guide the coherent light of the predetermined wavelength generated at the beam generation location (40) and coupled into the planar waveguide (6) in one or more predetermined directions along the surface of the planar waveguide (6) opposite to the surface facing the substrate (3).

18. Diffractometric sensor according to claim 16, further comprising an optical coupler (10) arranged on the planar waveguide and configured to couple the beam of coherent light generated at the beam generation location (40) into the planar waveguide (6) to impinge on the two interdigitated affinity gratings (2).

19. Diffractometric sensor according to claim 16, further comprising an optical decoupler (11) arranged on the planar waveguide (6) and configured to decouple the coherent light diffracted by the two interdigitated affinity gratings (2) from the planar waveguide (6) and direct it to the predetermined detection location (50).

20. Diffractometric sensor according to claim 16, further comprising a detector (5) for detecting the coherent light diffracted by the two interdigitated affinity gratings (2), the detector (5) being integrated in the planar waveguide (6) or in the substrate (3).

21. Diffractometric sensor according to claim 16, further comprising a light source (4) for generating the beam of coherent light of the predetermined wavelength, the light source (4) being integrated in the planar waveguide (6) or in the substrate (3).

22. Diffractometric sensor according to claim 15, wherein the resonant waveguiding structure arranged on the surface of the substrate comprises a metal layer, and wherein the two interdigitated affinity gratings are arranged on a surface of the metal layer opposite to the surface of the metal layer facing the substrate.

23. Diffractometric sensor according to claim 1, wherein the affinity elements of the first type contained in the first unit cells of the first grating and the affinity elements of the second type contained in the second unit cells of the second grating are obtained using bioorthogonal coupling chemistries.
